(19) United States
(12) Patent Application Publication
(10) Pub. No.: US 2025/0375237 A1
(43) Pub. Date: Dec. 11, 2025

(54) ABLATION CATHETERS WITH PRESSURE SENSOR TO TREAT VARICOSE VEINS

(71) Applicant: Boston Scientific Medical Device Limited, Galway (IE)

(72) Inventors: Yinghua Wang, Shanghai (CN); Mengxiang Luo, Shanghai (CN); Mingfeng Frank Xie, Shanghai (CN); Cheng Zhang, Shanghai (CN); Longsheng Cai, Shanghai (CN); Pan Linshanzi, Shanghai (CN)

(21) Appl. No.: 18/881,673

(22) PCT Filed: Jul. 13, 2023
(86) PCT No.: PCT/EP2023/069527

§ 371 Date: Jan. 6, 2025

(30) Foreign Application Priority Data
Jul. 14, 2022 (CN) 202210833123.8

(51) Int. Cl. A61B 18/08; A61B 17/00; A61B 18/00; A61B 90/00

(52) U.S. Cl. CPC A61B 18/082; A61B 2017/00292; A61B 2018/00404; A61B 2018/00577; A61B 2090/065

(57) ABSTRACT

A catheter for use in varicose vein treatment may include a handle, an elongated shaft connected to the handle, and a heating element disposed near the distal end of the shaft. In some embodiments, the heating element may include a plurality of pressure sensors circumferentially offset from one another, each pressure sensor being configured to generate an output signal indicative of pressure applied thereto by surface of target blood vessel.

### TECHNICAL FIELD

The present disclosure pertains to medical devices, systems, and methods for providing a therapeutic heat treatment. More particularly, the present disclosure pertains to medical devices, systems and methods for providing therapeutic heat treatments to venous diseases.

### BACKGROUND

Therapeutic heat treatment can be used to treat a wide variety of medical conditions such as tumors, fungal growth, etc. Heat treatments can be used for treating medical conditions alongside other therapeutic approaches or as a standalone therapy. Heat treatment provides localized heating and thus does not cause any cumulative toxicity in contrast to other treatment methods such as drug-based therapy, for example.

One exemplary clinical application of therapeutic heat treatment is in the treatment of chronic venous diseases such as varicose veins, which may become enlarged and/or tortuous due to one or more pathological conditions. Application of sufficient thermal energy via an intravascular device can treat varicose veins by constricting or occluding the target veins.

There is a continuing need for improved devices and methods to provide focused, controlled thermal energy for thermally treating chronic venous conditions such as varicose veins while minimizing or eliminating effects on surrounding healthy tissue.

### SUMMARY

In Example 1, a device for treating varicose veins includes an elongated catheter. The elongated catheter may include an elongated shaft defining a longitudinal axis having a proximal end and a distal end, a heating element disposed near the distal end of the elongated shaft, and a plurality of pressure sensors longitudinally spaced from one another along the shaft. The shaft may be sized and configured such that the distal end can be inserted into a target blood vessel. The heating element may include a coil member having a plurality of first windings about the shaft in a first direction, wherein a plurality of openings in the plurality of first windings are defined along the length of the heating element. Each of the pressure sensors may be located on the shaft within a respective one of the openings in the plurality of first windings, and wherein adjacent pressure sensors are circumferentially offset from one another, each pressure sensor being configured to generate an output signal indicative of pressure applied thereto by surface of target blood vessel.

In Example 2, the device of Example 1, wherein the coil member further includes a plurality of second windings about the shaft in a second direction different than the first direction, wherein at least some of the plurality of second windings cross over the plurality of first windings at locations spaced along a length of the heating element, and wherein at least some of the openings are defined between the plurality of first windings and the plurality of second windings.

In Example 3, the device of either of Examples 1 or 2, wherein the plurality of pressure sensors include three pressure sensors; wherein two adjacent pressure sensors of the plurality of pressure sensors are circumferentially offset by an offset degree related to N from one another.

In Example 4, the device of Example 1, wherein the plurality of pressure sensors include a first pressure sensor pair and a second sensor pair, wherein the first sensor pair includes a first pressure sensor and a second pressure sensor adjacent to the first pressure sensor, wherein the second pressure sensor is circumferentially offset by a first offset angle from the first pressure sensor, wherein the second sensor pair includes a third pressure sensor and a fourth pressure sensor adjacent to the third pressure sensor, wherein the fourth pressure sensor is circumferentially offset by a second offset angle from the third pressure sensor, wherein the second offset angle is equal to the first offset angle.

In Example 5, the device of Example 1, wherein the first and second plurality of windings are arranged to define a plurality of coil segments; wherein adjacent coil segments are longitudinally spaced from one another, defining one or more segment gaps between each adjacent coil segments along a length of the shaft; wherein the device further comprising a temperature sensor; wherein a temperature sensor is disposed within a segment gap of the one or more segments gaps; wherein at least one pressure sensor of the plurality of pressure sensors is disposed in an opening within a coil segment.

In Example 6, the device of Example 1, wherein the plurality of pressure sensors include six pressure sensors.

In Example 7, the device of Example 6, wherein two adjacent pressure sensors of the plurality of pressure sensors are circumferentially offset by 60 degrees from one another.

In Example 8, the device of Example 6, wherein two adjacent pressure sensors of the plurality of pressure sensors are circumferentially offset by 120 degrees from one another.

In Example 9, the device of any of Examples 1-8, wherein the plurality of pressure sensors include at least one selected from a group consisting of a piezoelectric pressure sensor, a capacitive pressure sensor, an inductive pressure sensor, a strain gauge pressure sensor, and a potentiometric pressure sensor.

In Example 10, the device of any of Examples 1-8, wherein the heating element is controlled to deliver ablative energy when an output signal indicative of pressure generated by one pressure sensor of the plurality of pressure sensors is greater than a predetermined threshold.

In Example 11, a device for treating varicose veins includes an energy generator configured to generate an electric signal; a controller operatively connected to the energy generator to control the generation of the electric signal; and an elongated catheter connected to the energy generator. The elongated catheter includes an elongated shaft defining a longitudinal axis having a proximal end and a distal end, the shaft being sized and configured such that the distal end can be inserted into a target blood vessel; a heating element disposed near the distal end of the elongated shaft; and a plurality of pressure sensors longitudinally spaced from one another along the shaft. The heating element may include a first coil member having a first plurality of windings about the shaft, wherein one or more first openings in the first plurality of windings are defined along the length of the first coil member; and a second coil member having a second plurality of windings about the shaft, wherein one or more second openings in the second plurality of windings are defined along the length of the second coil member. Each pressure sensor of the plurality of pressure sensors may be located on the shaft within a respective opening of the first openings in the first plurality of windings or the second openings in the second plurality of windings, and wherein at least two adjacent pressure sensors are circumferentially offset from one another, each pressure sensor being configured to generate an output signal indicative of pressure applied thereto by a surface of the target blood vessel. In some embodiments, the first and second coil members are each operatively connected to the energy generator and configured to generate thermal energy when the electric signal generated by the energy generator is delivered thereto.

In Example 12, the device of Example 11, wherein the heating element further comprises a third coil member comprising a third plurality of windings about the shaft, wherein one or more third openings in the third plurality of windings are defined along a length of the third coil member; wherein one or more of the plurality of pressure sensors are located on the shaft within one or more of the third openings,

In Example 13, the device of Example 11, wherein the controller is configured to adjust the current generated by the energy generator based on the output signal indicative of pressure applied thereto generated by each pressure sensor of the plurality of pressure sensors.

In Example 14, the device of any of Examples 11-13, wherein the controller is configured to control current generated by the energy generator to be selectively delivered to one or both of the first and second coil members.

In Example 15, the device of any of Examples 11-14, further comprising a temperature sensor disposed on the shaft within an opening of the first openings or the second openings, wherein the temperature sensor is longitudinally spaced from one of the plurality of pressures sensors along the shaft.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an exemplary ablation device for treating chronic venous diseases, e.g., varicose veins, according to an embodiment of the present disclosure.

FIG. 2A is a schematic illustration of an exemplary ablation catheter including a connector for treating chronic venous diseases, e.g., varicose veins, according to an embodiment of the present disclosure.

FIG. 2B is a schematic cross-sectional view of the connector of the exemplary ablation catheter of FIG. 2A, according to embodiments of the present disclosure.

FIGS. 3A-3C are schematic elevation view, partial blown-up view, and partial cross-sectional view, respectively, of the distal end portion of an ablation catheter, according to embodiments of the present disclosure.

FIG. 4 is a schematic illustration of the distal end portion of an ablation catheter, according to embodiments of the present disclosure.

FIG. 5 is a schematic illustration of the distal end portion of an ablation catheter, according to embodiments of the present disclosure.

FIG. 6 is a schematic illustration of the distal end portion of an ablation catheter, according to embodiments of the present disclosure.

FIG. 7 is a schematic illustration of the distal end portion of an ablation catheter, according to embodiments of the present disclosure.

FIGS. 8A-8C are schematic elevation view, partial cross-sectional view, and projection view respectively, of the distal end portion of an ablation catheter, according to embodiments of the present disclosure.

FIG. 9 is a schematic illustration of the distal end portion of an ablation catheter, according to embodiments of the present disclosure.

FIG. 10 is a schematic illustration of the distal end portion of an ablation catheter, according to embodiments of the present disclosure.

FIGS. 11A-11B are schematic illustrations of a portion of an ablation catheter for use in a target blood vessel in a patient for treatment of varicose veins, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Therapeutic heat treatment can be used to treat a wide variety of medical conditions including chronic venous diseases such as varicose veins, which may become enlarged and/or tortuous due to one or more pathological conditions. Application of sufficient thermal energy via an intravascular device can treat varicose veins by constricting or occluding the target veins.

An exemplary catheter for use in varicose vein treatment may include a handle, an elongated shaft connected to the handle, and a heating element disposed near the distal end of the shaft. In some embodiments, the heating element may include coils receiving currents (e.g., alternating currents, direct currents) delivered by an energy generator to generate and deliver thermal ablative energy. In certain embodiments, the heating element may include coils receiving electrical signals (e.g., radiofrequency alternating currents) generated by an energy generator to generate and deliver radiofrequency ablative energy.

As mentioned above, there is a continuing need for improved devices and methods to provide focused, controlled thermal energy for thermally treating chronic venous conditions such as varicose veins while minimizing or eliminating effects on surrounding healthy tissue. For example, physicians needs to ensure that the shaft including the heating element fits into a vein and has good contact with the target treatment site within the vein. Insufficient contact between the vein wall and the heating element may result in loss of efficiency in treating the disease and elongate treatment time, or ineffective treatment result. Therefore, physicians may benefit from real-time localized measurement of pressure within the target blood vessel to better determine treatment perimeters (e.g., temperature, time, etc.) for better treatment result and efficiency.

Some embodiments of the present disclosure describe a catheter with an elongated shaft defining a longitudinal axis having a proximal end and a distal end and a heating element disposed near the distal end of the shaft. In some embodiments, the heating element may include a coil member having a plurality of first windings about the shaft in a first direction, and a plurality of openings in the plurality of first windings are defined along the length of the heating element. In an exemplary embodiment, the catheter may further include a plurality of pressure sensors longitudinally spaced from one another along the shaft, wherein each of the pressure sensors is located on the shaft within a respective one of the openings in the plurality of first windings, and wherein adjacent pressure sensors are circumferentially offset from one another, each pressure sensor being configured to generate an output signal indicative of pressure applied thereto by surface of target blood vessel.

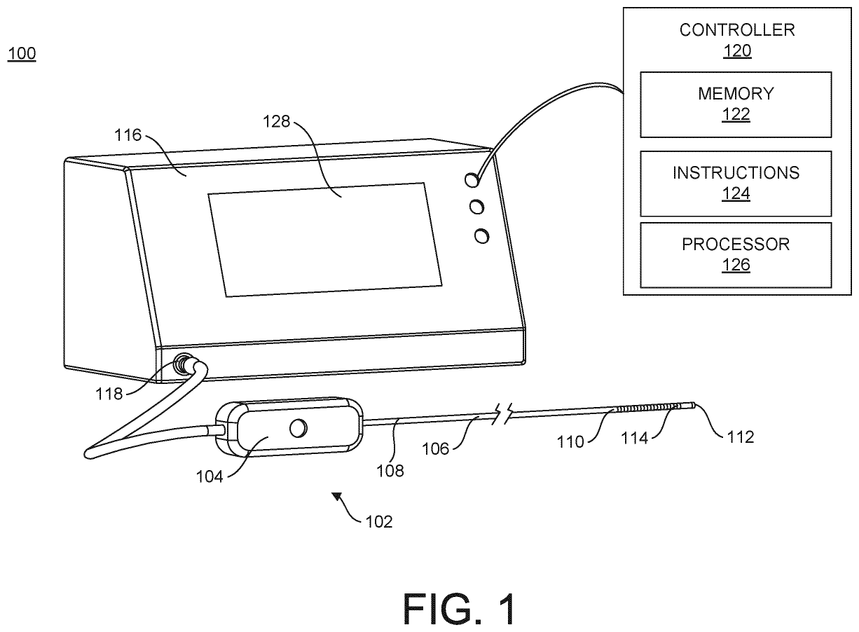

FIG. 1 is a schematic illustration of an exemplary ablation device 100 for treating chronic venous diseases, e.g., varicose veins, according to an embodiment of the present disclosure. The ablation device 100 includes an ablation catheter 102 including a handle 104, an elongated shaft 106 having a proximal end 108 and a distal end portion 110 terminating at a distal end 112, and a heating element 114 disposed near the distal end 112 of the elongated shaft 106. The shaft 106 is sized and configured such that the distal end 112 may be inserted into a target blood vessel. The heating element 114 is configured to deliver ablative energy (e.g., radiofrequency energy, thermal energy) to walls of a target blood vessel.

The device 100 may include an energy generator 116 electrically coupled to the handle 104 via a connector 118 and configured to generate energy by delivering an electric signal (e.g., currents, radiofrequency alternating currents). A controller 120 is operatively connected to the energy generator 116 to control the generation of the electric signal. The controller 120 can be implemented using firmware, integrated circuits, and/or software modules that interact with each other or are combined together. For example, the controller 120 may include memory 122 storing computer-readable instructions/code 124 for execution by a processor 126 (e.g., microprocessor) to perform aspects of embodiments of methods discussed herein.

According to certain embodiments, the heating element 114 employs structural features and/or components to improve the clinical performance as well as enhance the manufacturability of the ablation catheter 102. In some embodiments, the heating element 114 may include two or more coils with windings in different directions about the shaft 106, where the two or more coils cross over each other at a plurality of locations along the shaft 106, for example, resulting in a larger diameter of the heating element 114. In certain embodiments, the two or more coils may be made of individual conductor wires, where the controller 120 is configured to adjust power of treatment by selectively delivering current and/or delivering specific currents (e.g., different currents) generated by the energy generator 116 to the two or more conductor wires. In some embodiments, the heating element 114 includes a plurality of coil segments, where one or more coil segments are configured to be individually controlled and/or addressed. In certain embodiments, one or more coil segments include two or more coils with windings in one or more directions. In some embodiments, one or more coil segments include two or more coils having crossovers to each other at one or more locations.

In some embodiments, the controller 120 may be configured to communicate with various components of the device 100 and generate a graphical user interface (GUI) to be displayed via a display 128. The controller 120 may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the device 100.

In some embodiments, the controller 120 includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 122 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 122 stores computer-executable instructions for causing a processor (e.g., the controllers 120) to implement aspects of embodiments of system components discussed herein and/or to perform aspects of embodiments of methods and procedures discussed herein.

The computer-executable instruction 124 may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory 122 may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

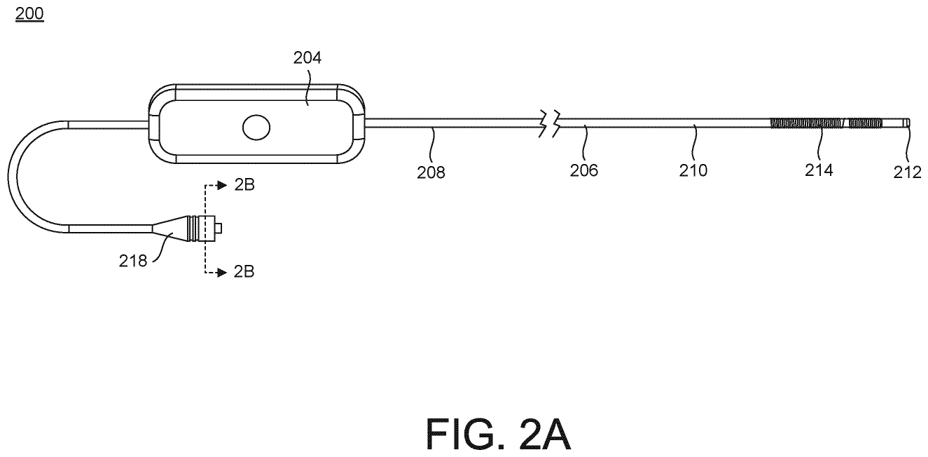

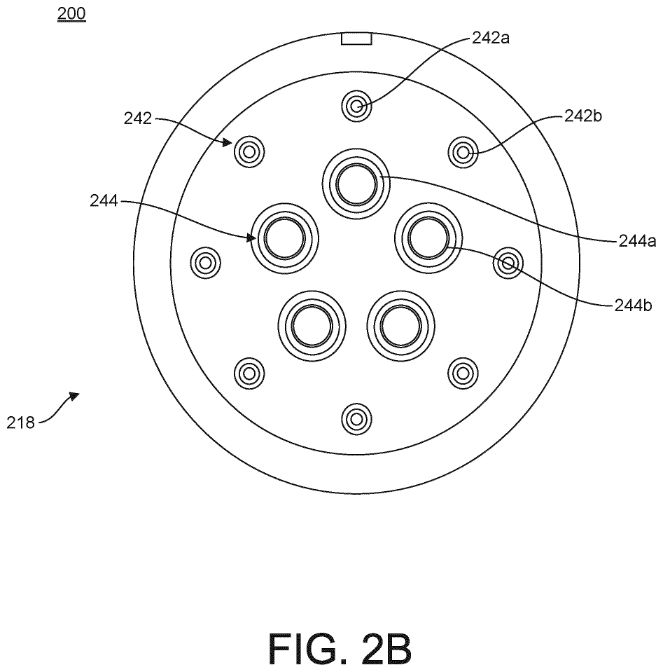

FIG. 2A is a schematic illustration of an exemplary ablation catheter 200 including a connector 218 (similar to the connector 118 as shown in FIG. 1) for treating chronic venous diseases, e.g., varicose veins; FIG. 2B is a schematic cross-sectional view of the connector 218 of the exemplary ablation catheter 200 along the cross-sectional indicator lines 2B-2B of FIG. 2A, according to embodiments of the present disclosure.

As shown, the ablation catheter 200 includes a handle 204, an elongated shaft 206 having a proximal end 208 and a distal end portion 210 terminating at a distal end 212, and a heating element 214 disposed near the distal end 212 of the elongated shaft 206. The shaft 206 is sized and configured such that the distal end 212 may be inserted into a target blood vessel. The heating element 214 is configured to deliver ablative energy (e.g., radiofrequency energy, thermal energy) to the wall of a target blood vessel.

In some embodiments, the connector 218 includes pins of different sizes 242 (including e.g., pins 242a, 242b) and 244 (including e.g., pins 244a, 244b). The pins 242 are relatively smaller than pins 244, and are configured to transfer electric signals (e.g., the electric signal generated by the energy generator 116 in FIG. 1). Exemplary electric signals may include thermocouple signals or pressure signals. The pins 244 are relatively larger compared to pins 242, and may be configured to allow current to pass from an energy generator (e.g., the energy generator 116 in FIG. 1) to generate heat on the heating element 214. One of the pins 244 may be used as a pin connected to ground (i.e., a ground pin). In some embodiments, where the heating elements include multiple heating segments (e.g., coil segments), the ground pin may be used as a common ground pin by multiple heating segments.

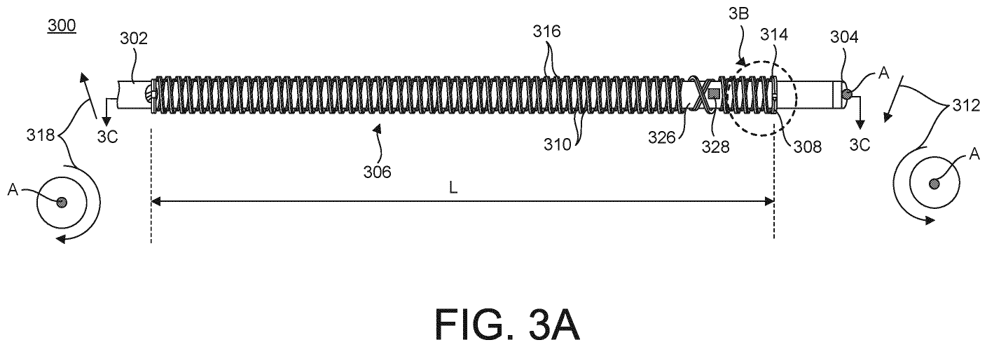

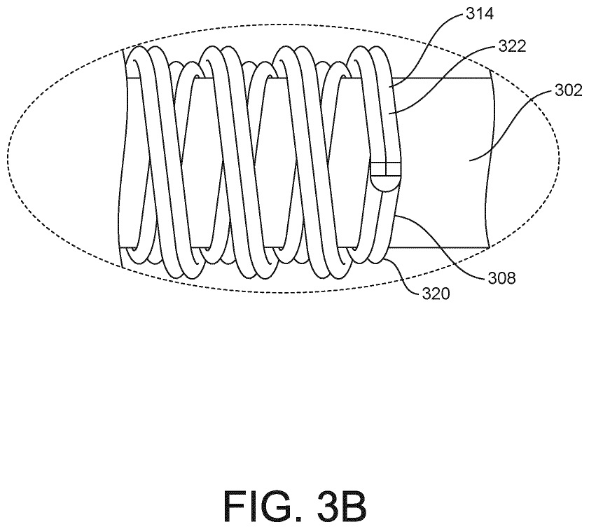

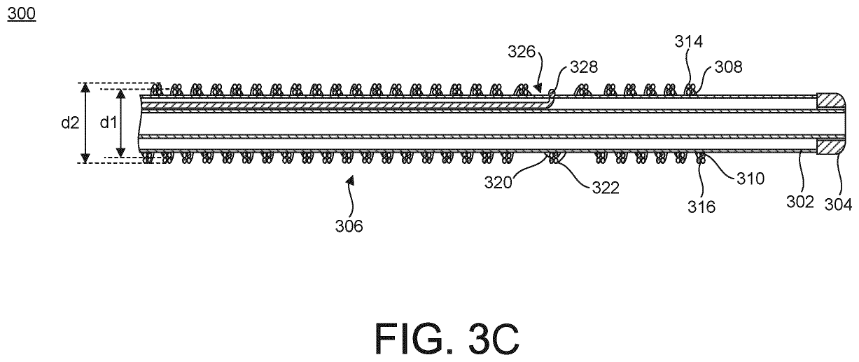

FIGS. 3A-3C include a schematic elevation view, a partial blown-up view, and a partial cross-sectional view, respectively, of an example of a distal end portion 300 of an ablation catheter, according to embodiments of the present disclosure.

In some embodiments, the distal end portion 300 of the ablation catheter (e.g., the ablation catheter 102 in FIG. 1, the ablation catheter 200 in FIG. 2A) includes a part of an elongated shaft 302 terminating at a distal end 304, also referred to as a distal end portion of the shaft 302, and a heating element 306 disposed near the distal end 304 of the elongated shaft 302. The shaft 302 is sized and configured such that the distal end 304 may be inserted into a target blood vessel.

The heating element 306 includes a first heating coil 308 having a plurality of first windings 310 in a first direction 312 (indicated by arrows around a reference point A), and a second heating coil 314 having a plurality of second windings 316 in a second direction 318 (indicated by arrows around a reference point A). As shown, the first direction 312 is different from the second direction 318, and the second windings 316 cross over the first windings 310 at locations spaced along a length (L) of the distal end portion 300 of the shaft 302. In some embodiments, the length (L) may be from about 2 cm to about 10 cm long. In some embodiments, the length (L) may be from about 3 cm to about 8 cm long. In an exemplary embodiment, the length L may be from about 5 cm to about 7 cm long. The windings 310 and 316 may be wrapped around the shaft 302 using a winding machine to achieve tighter and smoother heating coils 308 and 314 around the shaft 302.

FIG. 3B is a partial blown-up view of an example of a distal end portion 300 of an ablation catheter, indicated by circle 3B of FIG. 3A. As shown, the coil 308 includes a conductor wire 320, and the coil 314 may include a conductor wire 322. In some embodiments, the conductor wires 320 and 322 may be the same wire. In certain embodiments, the conductor wires 320 and 322 may be different wires. The conductor wires 320 and 322 may be single-filar (as shown) or multi-filar (not shown). In embodiments, the conductive wires 320 and 322 have an insulation cover respectively, such that the conductive wire 320 is electrically isolated from the conductive wire 322 when the catheter is in use. In an exemplary embodiment, the insulation cover may be polyurethane or polyimide. In certain embodiments, the conductor wire 320 and 322 may include single-filar wires, each symmetrically folded and wound on the elongated shaft 302.

In some instances, the pitch (i.e. distance between the midpoints of two adjacent wires) between wires 320 and 322 may be the same. In some instances, the pitch between the wires 320 and 322 may be different. In some embodiments, the wires 320 and 322 may be wound in the same direction (i.e., both clockwise, or both counterclockwise). In some embodiments, the wires 320 and 322 may be wound in opposite directions.

FIG. 3C is a partial cross-sectional view of an example of a distal end portion 300 of an ablation catheter, indicated by arrows 3C of FIG. 3A. Because of the crossover between the coils 308 and 314, the diameter of the heating element 306 is increased from d1 to d2, as shown in FIG. 3C. The difference between d1 and d2 is equal to or greater than the thickness of the second heating coil 314. In some embodiments, the resulting diameter of the heating element 306 may be from about 1 mm to about 4 mm. In an exemplary embodiment, the resulting diameter of the heating element 306 may be from about 2 mm to about 3 mm. In some embodiments, the coils 308 and 314 are operatively connected to an energy generator (e.g., the energy generator 116 in FIG. 1) and configured to generate thermal energy in response to receiving an electric signal (e.g., radiofrequency currents) from the energy generator.

In some embodiments, as shown in FIGS. 3A-3C, the conductor wires 320 and 322 may be single-filar. In some embodiments, the conductor wires 320 and 322 may be multi-filar (not shown). In an exemplary embodiment, the conductor wire 320 may include one filar symmetrically folded and wound on the elongated shaft 302, and the conductor wire 322 may include one filar symmetrically folded and wound on the elongated shaft 302. The number of filars in conductor wires 320 and 322 may be the same or different depending on the desirable diameter for a specific treatment site, and may be adjusted by either including more or fewer filars in the first heating coil 308 and/or the second heating coil 314.

The crossover design makes it possible to achieve any desirable diameter of the heating element 306 through simply adjusting the number of filers in each of the conductor wires. This allows ease of manufacturing by eliminating the need to make different sized shafts (e.g., elongated shaft 302 in FIGS. 3A-3B). As veins may become tortuous due to chronic venous diseases, it is not easy for operators to insert the distal end portion 300 of an ablation catheter into the target vein. Placement of the heating element 306 on the distal end portion 300 to a specific treatment site may become increasingly difficult if the catheter is too stiff. Increasing flexibility of the catheter makes it easier for the distal end portion 300 to go through tortuous veins and arrive at target treatment site, and may also reduce the operation time. In addition, the crossover design may increase the diameter of catheter without increasing the diameter of flexible elongated shaft 302.

In addition, the conductor wires 320, 322 may be electrically isolated from one another, and each be controlled by a controller (e.g., the controller 120 in FIG. 1) to generate heat either individually or simultaneously. Thus the physician and/or the controller may have flexibility to make adjustments on how much heat is used for treatment depending on patient need and treatment progress.

In some embodiments, the conductor wires 320 and 322 are electrically connected in series, and would receive the same current from an energy generator (e.g., the energy generator 116 in FIG. 1) going through them. In some embodiments, the conductor wires 320 and 322 are electrically isolated from one another and are each individually addressable by an energy generator (e.g., the energy generator 116 in FIG. 1). When the wires 320 and 322 are electrically isolated from one another, a controller (e.g., the controller 120 in FIG. 1) may be configured to selectively deliver current generated by the energy generator to one or both of the first and second conductor wires.

In some embodiments, the heating element 306 includes a plurality of coil segments longitudinally spaced from one another along the length of the distal end portion, where each coil segment includes a portion of the first heating coil and a portion of the second heating coil. In some embodiments, the heating coils 308 and 314 are resistance heating coils.

In some embodiments, the electric signal generated by an energy generator (e.g., the energy generator 116 in FIG. 1) may be a radiofrequency alternating current and the heating coils 308 and 314 are configured to deliver radiofrequency ablative energy to target tissue. In certain embodiments, one or more ground pads are used with the heating coils 308 and 314 to deliver the radiofrequency ablative energy to a target vessel. In some embodiments, the heating coils 308 and 314 are configured to form bipolar electrodes to deliver the radiofrequency ablative energy to the target tissue or vessel. For example, the heating coils 308 and 314 include two or more coil segments where two of the coil segments form an electrode pair.

In some embodiments, an opening 326 may be formed along the length of the heating element 306, and a temperature sensor 328 may be disposed in the opening 326. Based on temperature or signals indicative of temperature measured by the temperature sensor 328, a controller (e.g., the controller 120 in FIG. 1) may be configured to adjust respective current or selectively deliver current to one or both of the conductor wires 320, 322. In some examples, if the measured temperature is too high, the controller may reduce the current generated by the energy generator. In certain examples, if the measured temperature is too high, the controller may deliver current generated by the energy generator to only one of the conductor wires 320, 322. In some examples, if the measured temperature is too low, the controller may increase the current generated by the energy generator. In certain examples, if the measured temperature is too low, the controller may deliver current generated by the energy generator to both of the conductor wires 320, 322.

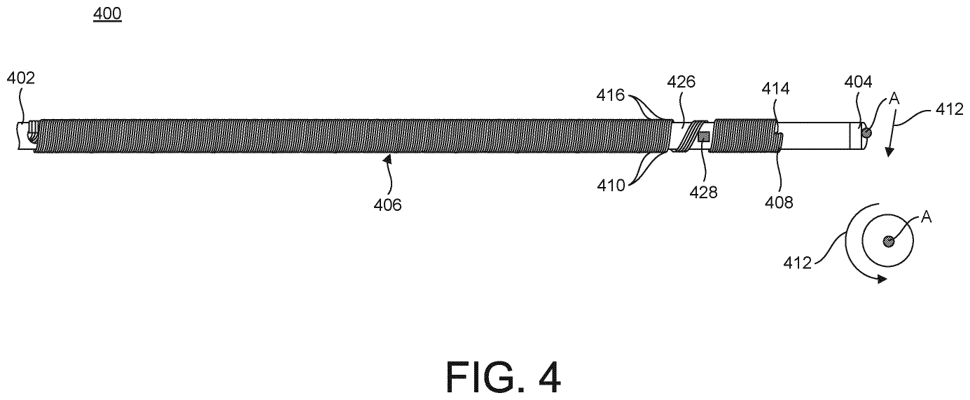

FIG. 4 is a schematic illustration of the distal end portion of the ablation catheter of FIG. 1, according to embodiments of the present disclosure. As shown, the distal end portion 400 includes part of an elongated shaft 402 terminating at a distal end 404, and a heating element 406 disposed near the distal end 404 of the elongated shaft 402. The shaft 402 is sized and configured such that the distal end 404 may be inserted into a target blood vessel.

The heating element 406 includes a first heating coil 408 having a plurality of first windings 410 in a direction 412, and a second heating coil 414 having a plurality of second windings 416 about the shaft 402 in the direction 412 (indicated by arrows around a reference point A) and co-radially with the first heating coil 408. In some embodiments, the coils 408 and 414 are operatively connected to an energy generator (e.g., the energy generator 116 in FIG. 1) and configured to generate thermal energy when a current supplied by the energy generator is delivered thereto. In some embodiments, the coils 408 and 414 are electrically isolated from one another and individually addressable by the energy generator.

In some embodiments, each of the coils 408 and 414 may include a single-filar conductor wire. In some embodiments, each of the coils 408 and 414 may include a multi-filar conductor wire. In certain embodiments, the first and second heating coils 408, 414 may include single-filar wires, each symmetrically folded and wound on the elongated shaft 402. In some embodiments, a controller (e.g., the controller 120 in FIG. 1) may be configured to selectively deliver current generated by the energy generator to one or both of the first and second conductor wires.

In some embodiments, an opening 426 may be formed along the length of the heating element 406, and a temperature sensor 428 may be disposed in the opening 426. Based on temperature or signals indicative of temperature measured by the temperature sensor 428, a controller (e.g., the controller 120 in FIG. 1) may be configured to adjust respective current or selectively deliver current to one or both of the conductor wires of the coils 408 and 414. In some examples, if the measured temperature is too high, the controller may reduce the current generated by the energy generator. In certain examples, if the measured temperature is too high, the controller may deliver current generated by the energy generator to only one of the conductor wires of the coils 408 and 414. In some examples, if the measured temperature is too low, the controller may increase the current generated by the energy generator. In certain examples, if the measured temperature is too low, the controller may deliver current generated by the energy generator to both of the conductor wires of the coils 408 and 414.

In some embodiments, the first and second heating coils 408 and 414 are resistance heating coils. In some embodiments, the electric signal generated by an energy generator (e.g., the energy generator 116 in FIG. 1) may be a radiofrequency alternating current and the first and second heating coils 408 and 414 are configured to deliver radiofrequency ablative energy to target tissue.

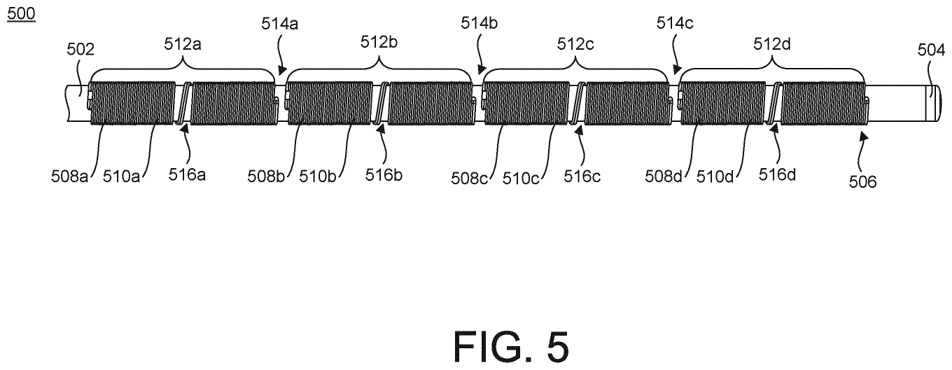

FIG. 5 is a schematic illustration of the distal end portion 500 of an ablation catheter, according to embodiments of the present disclosure. As shown, the distal end portion 500 includes part of an elongated shaft 502 terminating at a distal end 504, and a heating element 506 disposed near the distal end 504 of the elongated shaft 502. The shaft 502 is sized and configured such that the distal end 504 may be inserted into a target blood vessel.

As shown, the heating element 506 may include one or more coils 508a-d, each having a plurality of windings 510a-d about the shaft 502. Each of the plurality of windings 510 defines a coil segment (e.g., 512a-d), and one or more segment gaps 514a-c in between each of the adjacent coil segments 512a-d. The windings 510 may be wrapped around the shaft 502 using a winding machine to achieve tighter and smoother coils 508 around the shaft 502. Using a winding machine may also help ensure the position of each coil segments (e.g., 512a-d).

The segmented design creates the one or more segment gaps 514a-c, thus increasing the flexibility of an ablation catheter (e.g., the ablation catheter 102 in FIG. 1, the ablation catheter 200 in FIG. 2A) and minimizing potential undesirable harm to vessel walls during treatment. Each of the coil segments 512a-d may be of the same length. In some embodiments, the coil segments 512a-d may be wounded by the same wire. In some embodiments, each of the coil segments 512a-d may be wounded by different, separate wires. In certain embodiments, when each of the coil segments 512a-d are wounded by different wires, a part of or all of the coil segments 512a-d are individually addressable by an energy generator (e.g., the energy generator 116 in FIG. 1) and/or controllable by a controller (e.g., the controller 120 in FIG. 1). For example, the coil segment 512a may be supplied with an ablation current while the coil segment 512b is not supplied with an ablation current. Having part or all of the coil segments 512a-d individually addressable and controllable, a current may be selectively applied to each of the coil segments 512a-d to create electrical pathways of different lengths, thus selectively change the effective length of heat treatment.

In some embodiments, the coils 508a-d are operatively connected to an energy generator (e.g., the energy generator 116 in FIG. 1) and configured to generate thermal energy when a current supplied by the energy generator is delivered thereto. In some embodiments, the coils 508a-d are individually addressable by the energy generator. In some embodiments, each of the coils 508a-d may include a single-filar conductor wire. In some embodiments, each of the coils 508a-d may include a multi-filar conductor wire. In some embodiments, a controller (e.g., the controller 120 in FIG. 1) may be configured to selectively deliver current generated by the energy generator to one or more of the conductor wires in the coils 508a-d.

In some embodiments, as shown, the plurality of windings 510a-d defining each of the coil segments 512a-d may include an opening 516a-d within the each of the coil segments 512a-d. In certain embodiments, one or more temperature sensors (e.g., the temperature sensor 328 or 428 in FIGS. 3A-B and FIG. 4) may be disposed in the openings 516a-d.

In some embodiments, the coils 508a-d are resistance heating coils. In some embodiments, the electric signal generated by an energy generator (e.g., the energy generator 116 in FIG. 1) may be a radiofrequency alternating current and the coils 508a-d are configured to deliver radiofrequency ablative energy to target tissue.

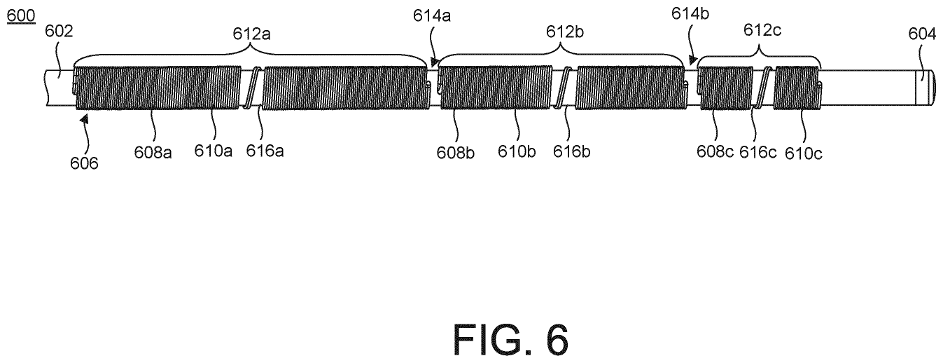

FIG. 6 is a schematic illustration of the distal end portion 600 of an ablation catheter, according to embodiments of the present disclosure.

As shown, the distal end portion 600 includes part of an elongated shaft 602 terminating at a distal end 604, and a heating element 606 disposed near the distal end 604 of the elongated shaft 602. The shaft 602 is sized and configured such that the distal end 604 may be inserted into a target blood vessel.

As shown, the heating element 606 may include one or more coils 608a-c, each having a plurality of windings 610a-c about the shaft 602. Each of the plurality of windings 610 defines a coil segment 612a-c, and one or more segment gaps 614a-b in between each of the adjacent coil segments 612a-d. The segmented design creates the one or more segment gaps 614a-b. In certain embodiments, the shaft 602 includes a flexible material and with the one or more segment gaps 614a-b, the heating element 606 and the distal end portion 600 of an ablation catheter has increased flexibility, for example, to minimize potential undesirable harm to vessel walls during treatment. Each of the coil segments 612a-c may be of different lengths. For example, the coil segment 612a has a length different from a length of the coil segment 612b. As an example, the coil segment 612b has a length different from a length of the coil segment 612c.

In some embodiments, the plurality of windings may define from 2-8 coil segments, each coil segments may be from about 1 cm to about 5 cm long. In some embodiments, the plurality of windings may define from 3-6 coil segments, each coil segments may be from about 1 cm to about 3 cm long. In an exemplary embodiment, for example as shown in FIG. 5, the length of the coil segments may be the same, the plurality of windings 510a-d includes 4 coil segments 512a-d, and each coil segment may be from about 1.4 cm to about 2.3 cm long. In an exemplary embodiment, for example as shown in FIG. 6, the length of the coil segments may be different, the plurality of windings 610a-c includes 3 coil segments 612a-c, and each coil segment may be from about 1 cm to about 4 cm long.

In some embodiments, the coils 608a-c are operatively connected to an energy generator (e.g., the energy generator 116 in FIG. 1) and configured to generate thermal energy when a current supplied by the energy generator is delivered thereto. In some embodiments, the coils 608a-c are individually addressable by the energy generator. In certain embodiments, each of the coils 608a-c may include a single-filar conductor wire. In some embodiments, each of the coils 608a-c may comprise a multi-filar conductor wire. In some embodiments, a controller (e.g., the controller 120 in FIG. 1) may be configured to selectively deliver current generated by the energy generator to one or more of the conductor wires in the coils 608a-c.

In some embodiments, as shown, the plurality of windings 610a-c defining each of the coil segments 612a-c may include an opening 616a-c within the each of the coil segments 612a-c. In certain embodiments, one or more temperature sensors (e.g., the temperature sensor 328 or 428 in FIGS. 3A-B and FIG. 4) may be disposed in the openings 616a-c. In certain embodiments, the coils 608a-c are resistance heating coils. In some embodiments, the electric signal generated by an energy generator (e.g., the energy generator 116 in FIG. 1) may be a radiofrequency alternating current and the coils 608a-c are configured to deliver radiofrequency ablative energy to target tissue.

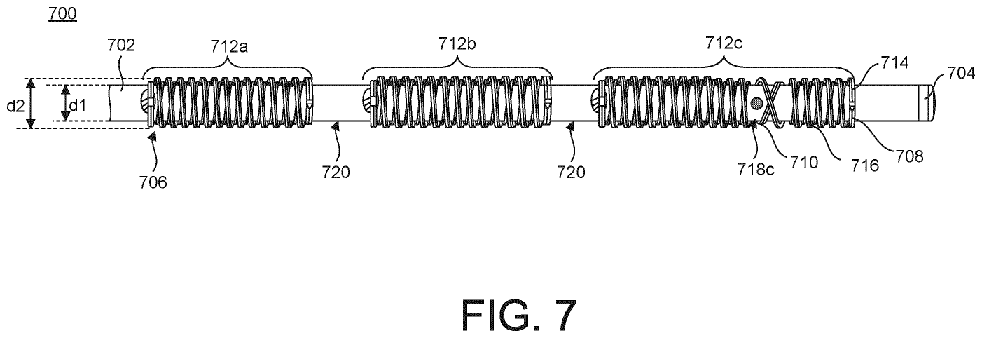

FIG. 7 is a schematic illustration of the distal end portion of an ablation catheter, according to embodiments of the present disclosure. As shown, the distal end portion 700 includes part of an elongated shaft 702 terminating at a distal end 704, and a heating element 706 disposed near the distal end 704 of the elongated shaft 702. The shaft 702 is sized and configured such that the distal end 704 may be inserted into a target blood vessel. In some examples, the distal end 704 has a diameter between two (2) millimeters and three (3) millimeters. In certain examples, the distal end 704 has a diameter between one (1) millimeter and five (5) millimeters. The heating element 706 may include one or more coil segments 712a-c. In certain embodiments, with the specific diameter ranges, the distal end 704 and/or the heating element 706 is configured to be inserted into blood vessels for ablation.

In some embodiments, the heating element 706 includes a first heating coil 708 having a plurality of first windings 710 in a first direction, and a second heating coil 714 having a plurality of second windings 716 in a second direction. In some embodiments, the first direction may be different from the second direction, and the second windings 716 cross over the first windings 710 within the length of each of the coils 712a-c. Because of the crossover between the coils 708 and 714, the diameter of each of the coil segments 712a-c of the heating element 706 is increased from d1 to d2, as shown in FIG. 3B. The difference between d1 and d2 is equal to or greater than the thickness of the second heating coil 714.

In some embodiments, the coils 708 and 714 are operatively connected to an energy generator (e.g., the energy generator 116 in FIG. 1) and configured to generate thermal energy in response to receiving an electric signal (e.g., a current) from the energy generator. In some embodiments, the coils 708 are individually addressable by the energy generator.

In some embodiments, one or more of the coil segments 712 (e.g., coil segment 712c) may include an opening 718c within the coil segment 712c. In certain embodiments, one or more temperature sensors (e.g., the temperature sensor 328 or 428 in FIGS. 3A-B and FIG. 4) may be disposed in the opening 718c. In certain embodiments, the coils 712a-c are resistance heating coils. In some embodiments, the electric signal generated by an energy generator (e.g., the energy generator 116 in FIG. 1) may be a radiofrequency alternating current and the coils 712a-c are configured to deliver radiofrequency ablative energy to target tissue.

In some embodiments, the first and second plurality of windings 710, 716 are arranged to define a plurality of coil segments 712a-c, wherein adjacent coil segments (e.g., 712a-b, or 712b-c) are longitudinally spaced from one another, defining one or more segment gaps 720 between each adjacent coil segments along a length of the shaft 702. In certain embodiments, the heating element 706 is configured as a plurality of coil segments 712a-c longitudinally spaced from one another along the length of the heating element 706, and wherein each coil segment 712a-c includes a portion of the first heating coil 708 and a portion of the second heating coil 714.

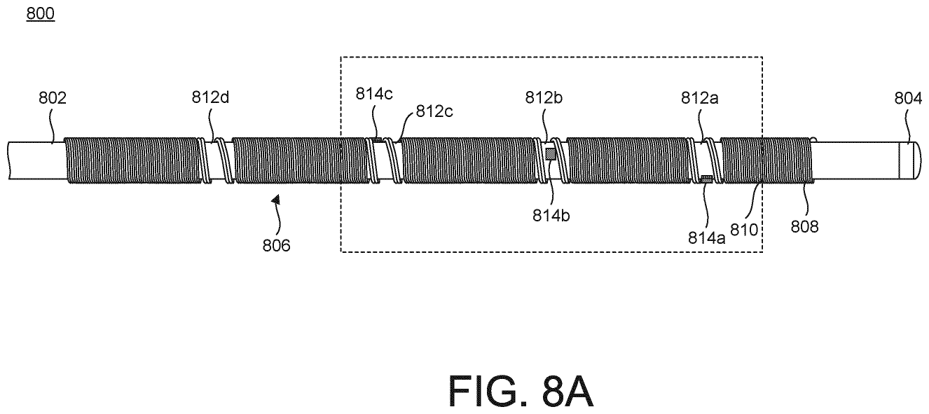

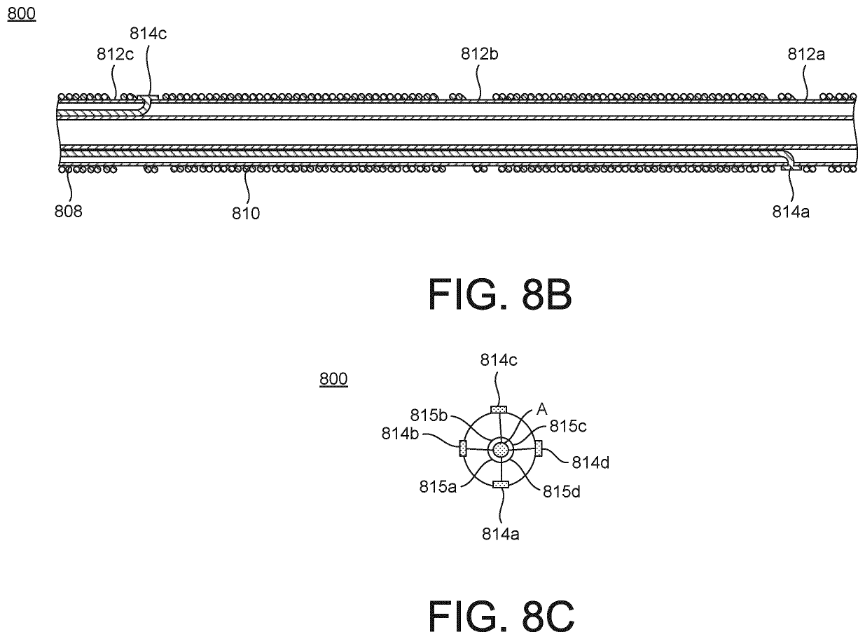

FIGS. 8A-8C are schematic elevation view, partial cross-sectional view, and projection view, respectively, of the distal end portion of an ablation catheter, according to embodiments of the present disclosure. As shown, the distal end portion 800 includes part of an elongated shaft 802 terminating at a distal end 804, and a heating element 806 disposed near the distal end 804 of the elongated shaft 802. The shaft 802 is sized and configured such that the distal end 804 may be inserted into a target blood vessel.

The heating element 806 includes a coil member 808 including a plurality of windings 810 about the shaft 802, and a plurality of openings 812a-d are defined in the plurality of windings 810 along the length of the heating element 806. In some embodiments, the coil member 808 is operatively connected to an energy generator (e.g., the energy generator 116 in FIG. 1) and configured to generate thermal energy when a current supplied by the energy generator is delivered thereto.

In some embodiments, a plurality of pressure sensors 814a-d are longitudinally spaced from one another along the shaft, and each of the pressure sensors 814a-d is located on the shaft within a respective one of the openings 812a-d in the plurality of first windings, and wherein adjacent pressure sensors are circumferentially offset from one another, each pressure sensor 814a-d being configured to generate an output signal indicative of pressure applied thereto by surface of target blood vessel. In certain embodiments, two adjacent pressure sensors have an circumferential offset angle between each other. In some examples, adjacent pressure sensors 814a and 814b include an offset angle 815a; adjacent pressure sensors 814b and 814c include an offset angle 815b; adjacent pressure sensors 814c and 814d include an offset angle 815c; and adjacent pressure sensors 814d and 814a include an offset angle 815d.

In some embodiments, the coil member 808 further includes a plurality of second windings (not shown) about the shaft 802 in a second direction different than the first direction, and at least some of the plurality of second windings cross over the plurality of first windings 810 at locations spaced along a length of the heating element 806. In some embodiments, at least some of the openings 812a-d are defined between the plurality of first windings 810 and the plurality of second windings. In certain embodiments, the pressure sensors are circumferentially distributed along the shaft with equal offset angles (e.g., angles 815a-d) between two adjacent pressure sensors. For example, in some embodiments, not shown, the plurality of pressure sensors include three pressure sensors, and each of the two adjacent pressure sensors of the three pressure sensors are circumferentially offset by 120 degrees from one another.

In an exemplary embodiment, as shown in FIGS. 8A-C, the plurality of pressure sensors include four pressure sensors 814a-d, and each of the two adjacent pressures sensors of the four pressure sensors 814a-d are circumferentially offset by 90 degrees from one another. In some embodiments, not shown, the plurality of pressure sensors include six pressure sensors, and each of the two adjacent pressure sensors of the six pressure sensors are circumferentially offset by 60 degrees from one another. In some embodiments, the plurality of pressure sensors include six pressure sensors, and each of the two adjacent pressure sensors of the six pressure sensors are circumferentially offset by 120 degrees from one another.

In some embodiments, the heating element 806 may further include a temperature sensor (not shown) disposed on the shaft 802. In some embodiments, the plurality of pressure sensors include at least one selected from a group consisting of a piezoelectric pressure sensor, a capacitive pressure sensor (i.e. a sensor that measures change of electrode impedance when an electrode contact a vein wall), an inductive pressure sensor, a strain gauge pressure sensor, a fiber optical pressure sensor, and a potentiometric pressure sensor. As will be appreciated by a skilled artisan, any type of sensor may be used here that may indicate the contact pressure between the sensor (e.g., sensors 814a-d) and the vein wall.

During treatment, the heating element 806 is controlled to deliver ablative energy when an output signal indicative of pressure generated by one pressure sensor of the plurality of pressure sensors is greater than a predetermined threshold. In some embodiments, the heating element 806 is controlled to deliver ablative energy when output signals indicative of pressure generated by two pressure sensors of the plurality of pressure sensors are greater than a predetermined threshold. In some embodiments, the heating element 806 is controlled to deliver ablative energy when output signals indicative of pressure generated by two adjacent pressure sensors of the plurality of pressure sensors are greater than a predetermined threshold. In some embodiments, the heating element 806 is controlled to deliver ablative energy when output signals indicative of pressure generated by all pressure sensors of the plurality of pressure sensors are greater than a predetermined threshold. The pressure sensors (e.g., 814a-d) are configured to monitor the pressure of the heating element 806 along the elongated shaft 802 such that an operator or controller (e.g., the controller 120 in FIG. 1) may estimate the seal off degree of the vein wall (i.e. the degree of vein constriction) during treatment to more accurately determine the course of treatment and adjust treatment plan accordingly.

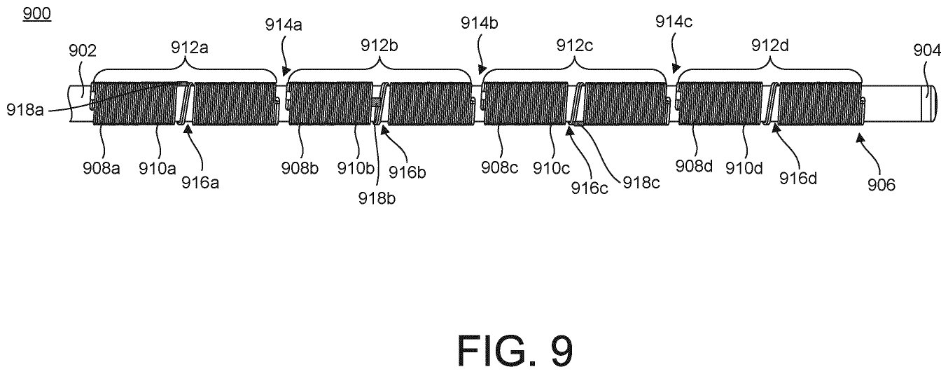

FIG. 9 is a schematic illustration of the distal end portion of an ablation catheter, according to embodiments of the present disclosure.

As shown, the distal end portion 900 includes part of an elongated shaft 902 terminating at a distal end 904, and a heating element 906 disposed near the distal end 904 of the elongated shaft 902. The shaft 902 is sized and configured such that the distal end 904 may be inserted into a target blood vessel.

As shown, the heating element 906 may include one or more coils 908a-d, each having a plurality of windings 910a-d about the shaft 902. Each of the plurality of windings 910 defines a coil segment 912a-d, and one or more segment gaps 914a-c in between each of the adjacent coil segments 912a-d. The segmented design creates the one or more segment gaps 914a-c, thus increasing the flexibility of the distal end portion 900 of an ablation catheter and minimizing potential undesirable harm to vessel walls during treatment. Each of the coil segments 912a-d may be of the same length. In certain embodiments, a part of or all of the coil segments 912a-d are individually addressable and/or controllable. For example, the coil segment 912a is supplied with an ablation current and the coil segment 912b is not supplied with an ablation current.

In some embodiments, the coils 908a-d are operatively connected to an energy generator (e.g., the energy generator 116 in FIG. 1) and configured to generate thermal energy when a current supplied by the energy generator is delivered thereto. In some embodiments, the coils 908a-d are individually addressable by the energy generator.

In some embodiments, each of the coils 908a-d may include a single-filar conductor wire. In some embodiments, each of the coils 908a-d may comprise a multi-filar conductor wire. In some embodiments, a controller (e.g., the controller 120 in FIG. 1) may be configured to selectively deliver current generated by the energy generator to one or more of the conductor wires in the coils 908a-d.

In some embodiments, as shown, the plurality of windings 910a-d defining each of the coil segments 912a-d may include an opening 916a-d within the each of the coil segments 912a-d. In certain embodiments, one or more temperature sensors (e.g., the temperature sensor 328 or 428 in FIGS. 3A-B and FIG. 4) may be disposed in the openings 916a-d.

In some embodiments, the coils 908a-d are resistance heating coils. In some embodiments, the electric signal generated by an energy generator (e.g., the energy generator 116 in FIG. 1) may be a radiofrequency alternating current and the coils 908a-d are configured to deliver radiofrequency ablative energy to target tissue or vessel.

In certain embodiments, the heating element 906 may include a plurality of sets of coil segments 912a-d with corresponding plurality of sets of pressure sensors 918a-d (pressure sensor 918d is not shown in FIG. 9), where a set of pressure sensors (e.g., a set of 3 pressure sensors, a set of 4 pressure sensors) cover a full circle with a set of coil segments (e.g., a set of 3 coil segments). In some examples, a sum of offset angles (e.g., four offset angles of 90 degree each, a set of offset angles of (90, 120, 90, 60) between adjacent pressure sensors of a set of pressure sensors is equal to 360 degree. In certain examples, a first set of pressure sensors and a second set of pressure sensors have a same adjacent offset angle pattern as each other. In some examples, a set of coil segment is activated (e.g., supplied with current(s)) when an output signal indicative of pressure generated by at least one pressure sensor of the set of pressure sensors is greater than a predetermined threshold. In some examples, a set of coil segment is deactivated (e.g., not supplied with current(s)) when none of the output signals indicative of pressure generated by the set of pressure sensors is greater than a predetermined threshold.

In an exemplary embodiment, at least one pressure sensor of the plurality of pressure sensors 918a-d is disposed in an opening (e.g., opening 916a, 916b, 916c, or 916d) within a coil segment (e.g., coil segment 912a, 912b, 912c, or 912d). In some embodiments, the heating element may further include a temperature sensor (e.g., the temperature sensor 328 or 428 in FIGS. 3A-B and FIG. 4) disposed within a segment gap of the one or more segment gaps 914a-c. The temperature sensor may be longitudinally spaced from one of the plurality of pressure sensors 918a-d, and circumferentially offset from one of the plurality of pressure sensors 918a-d.

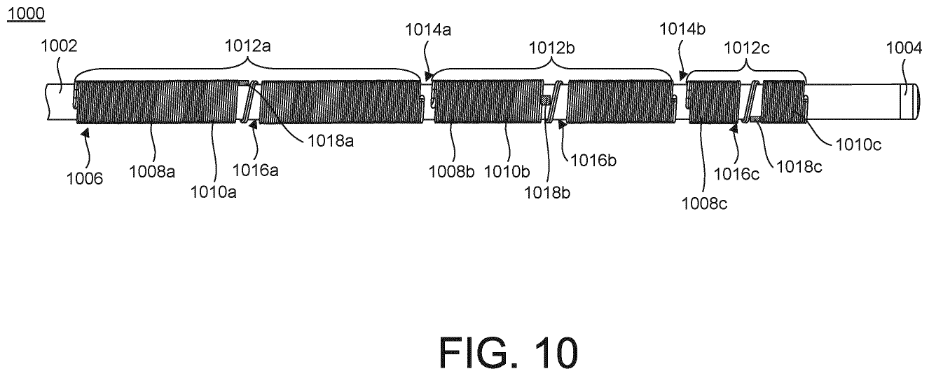

FIG. 10 is a schematic illustration of the distal end portion of an ablation catheter, according to embodiments of the present disclosure.

As shown, the distal end portion 1000 includes part of an elongated shaft 1002 terminating at a distal end 1004, and a heating element 1006 disposed near the distal end 1004 of the elongated shaft 1002. The shaft 1002 is sized and configured such that the distal end 1004 may be inserted into a target blood vessel.

As shown, the heating element 1006 may include one or more coils 1008a-c, each having a plurality of windings 1010a-c about the shaft 1002. Each of the plurality of windings 1010 defines a coil segment 1012a-c, and one or more segment gaps 1014a-b in between each of the adjacent coil segments 1012a-c. The segmented design creates the one or more segment gaps 1014a-b. In certain embodiments, the shaft 1002 includes a flexible material and with the one or more segment gaps 1014a-b, the heating element 1006 and the distal end portion 1000 of an ablation catheter has increased flexibility, for example, to minimize potential undesirable harm to vessel walls during treatment. Each of the coil segments 1012a-c may be of different lengths. For example, the coil segment 1012a has a length different from a length of the coil segment 1012b. As an example, the coil segment 1012b has a length different from a length of the coil segment 1012c.

In some embodiments, the coils 1008a-c are operatively connected to an energy generator (e.g., the energy generator 116 in FIG. 1) and configured to generate thermal energy when a current supplied by the energy generator is delivered thereto. In some embodiments, the coils 1008a-c are individually addressable by the energy generator. In certain embodiments, each of the coils 1008a-c may include a single-filar conductor wire. In some embodiments, each of the coils 1008a-c may comprise a multi-filar conductor wire. In some embodiments, a controller (e.g., the controller 120 in FIG. 1) may be configured to selectively deliver current generated by the energy generator to one or more of the conductor wires in the coils 1008a-c.

In some embodiments, as shown, the plurality of windings 1010a-c defining each of the coil segments 1012a-c may include an opening 1016a-c within the each of the coil segments 1012a-c. In certain embodiments, the coils 1008a-c are resistance heating coils. In some embodiments, the electric signal generated by an energy generator (e.g., the energy generator 116 in FIG. 1) may be a radiofrequency alternating current and the coils 1008a-c are configured to deliver radiofrequency ablative energy to target tissue or vessel.

In certain embodiments, the heating element 1006 may include a plurality of sets of coil segments 1012a-c with corresponding plurality of sets of pressure sensors 1018a-c, where a set of pressure sensors (e.g., a set of 3 pressure sensors) cover a full circle with a set of coil segments (e.g., a set of 3 coil segments). In some examples, a sum of offset angles (e.g., three offset angles of 120 degree each, a set of offset angles of (180, 120, 60)) between adjacent pressure sensors of a set of pressure sensors is equal to 360 degree. In some examples, a set of coil segment is activated (e.g., supplied with current(s)) when an output signal indicative of pressure generated by at least one pressure sensor of the set of pressure sensors is greater than a predetermined threshold. In some examples, a set of coil segment is deactivated (e.g., not supplied with current(s)) when none of the output signals indicative of pressure generated by the set of pressure sensors is greater than a predetermined threshold.

In an exemplary embodiment, at least one pressure sensor of the plurality of pressure sensors 1018a-c is disposed in an opening (e.g., opening 1016a, 1016b, or 1016c) within a coil segment (e.g., coil segment 1012a, 1012b, or 1012c). In some embodiments, the heating element 1006 may further include a temperature sensor (e.g., the temperature sensor 328 or 428 in FIGS. 3A-B and FIG. 4) disposed within a segment gap of the one or more segment gaps 1014a-b. The temperature sensor may be longitudinally spaced from one of the plurality of pressure sensors 1018a-c, and circumferentially offset from one of the plurality of pressure sensors 1018a-c. In certain embodiments, one or more temperature sensors (e.g., the temperature sensor 328 or 428 in FIGS. 3A-B and FIG. 4) may be disposed in the openings 1016a-c.

In some embodiments (for example as shown in FIG. 7), a heating element (e.g., the heating element 114 in FIG. 1) may include a first and second plurality of windings arranged to define a plurality of coil segments, where at least some of the second windings cross over the first windings along the length of the heating element, and adjacent coil segments are longitudinally spaced from one another, defining one or more segment gaps between each adjacent coil segments along a length of the shaft. In an exemplary embodiment, one or more openings are formed within one or more of the plurality of coil segments, and at least one pressure sensor of a plurality of pressure sensors is disposed in an opening within a coil segment. In some embodiments, the heating element may further include a temperature sensor disposed within a segment gap of the one or more segment gaps. The temperature sensor may be longitudinally spaced from one of the plurality of pressures sensors, and circumferentially offset from one of the plurality of pressures sensors.

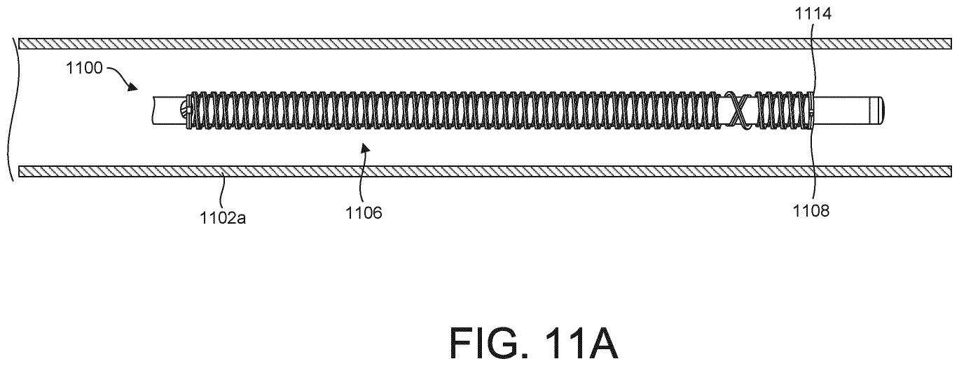

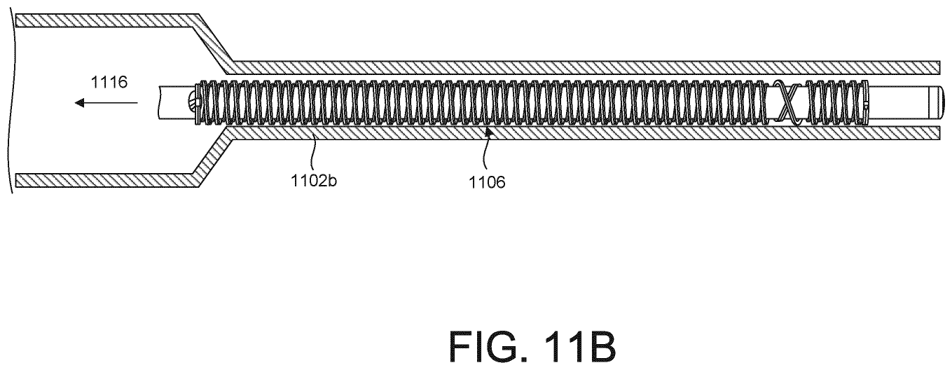

FIGS. 11A-11B are schematic illustrations of a portion of an ablation catheter for use in a target blood vessel in a patient for treatment of varicose veins, according to embodiments of the present disclosure.

In some embodiments, during endovenous thermal ablation procedure, an introducer sheath may be positioned inside a patient's target vein using ultrasonic guidance and standard vascular technique. An ablation catheter (e.g., the ablation catheter 102 in FIG. 1) may then be inserted into the target vein through the introducer sheath. In some circumstances, under ultrasonic guidance, tumescent anesthetic solution or saline may be injected into target vein segment to act as a heat sink that protects tissue from thermal injury, and improve thermal conductivity between the wall of target vein and the ablation catheter.

As shown in FIG. 11A, the distal end portion 1100 of an ablation catheter (e.g., the ablation catheter 102 in FIG. 1) is positioned in a target blood vessel 1102a. The ablation catheter may be introduced and positioned with an introducer sheath using ultrasonic guidance. As will be appreciated by a skilled artisan, any standard vascular technique may be used here to introduce and position the distal end portion 1100 of the ablation catheter into the target vein segment. The distal end portion 1100 may include a heating element 1106 having heating coils 1108 and 1114.

In some embodiments, during treatment, current may be applied to the heating coils 1108 and 1114 by a generator (e.g., the energy generator 116 in FIG. 1). The generator may include a radiofrequency generator that generates radiofrequency current to heat the heating coils 1108 and 1114. In some implementations, the ablation catheter may include a temperature sensor disposed along the length of a shaft of the catheter, and power delivery to the coils may be adjusted automatically by a controller (e.g., the controller 120 in FIG. 1) based on temperature or signals indicative of temperature measured by the temperature sensor. In some embodiments, the power delivery to the heating coils 1108 and 1114 may heat the heating coils 1108 and 1114 to about 80° C. to about 140° C. for treating varicose veins. In some embodiments, the power delivery to the heating coils 1108 and 1114 may heat the heating coils 1108 and 1114 to about 100° C. to about 130° C. for treating varicose veins. In some embodiments, the power delivery to the heating coils 1108 and 1114 may heat the heating coils 1108 and 1114 to about 120° C. for treating varicose veins.

A segment of the target blood vessel 1102a adjacent the heating coils 1108 and 1114 being treated will close (e.g., shrink, reduced in diameter) as the coils are heated up, shown as 1102b in FIG. 11B. External pressure may be applied as needed during treatment. After a certain section is treated (i.e. the section of the vein is closed), the catheter may be moved towards the venous access, as indicated by arrow 1116, and the process repeated until the entire vein is closed. The catheter and introducer sheath may then be removed after treatment is done. In some use cases, a diameter of the heating element 1106 is smaller than a diameter of blood vessel 1102a and the heating element 1106 can be moved close to the vessel wall during the treatment.

In some embodiments, a device for treating varicose vein may include an energy generator configured to generate a current; a controller operatively connected to the energy generator to control the generation of the current; and a catheter connected to the energy generator. The catheter may include a handle; an elongated shaft connected to the handle having a proximal end and a distal end portion terminating at a distal end, the shaft being sized and configured such that the distal end can be inserted into a target blood vessel; and a heating element disposed near the distal end of the elongated shaft, the heating comprising a first heating coil comprising a plurality of first windings about the shaft in a first direction, and a second heating coil comprising a plurality of second windings about the shaft in the first direction and co-radially with the first heating coil, wherein the first and second heating coils are each operatively connected to the energy generator and configured to generate thermal energy when the current supplied by the energy generator is delivered thereto, and wherein the first and second heating coils are electrically isolated from one another and individually addressable by the energy generator. In some embodiments, the first and second heating coils may each comprise a single-filar conductor wire. In some embodiments, the first and second heating coils may each comprise a multi-filar conductor wire. In some embodiments, the first and second heating coils may be resistance heating coils, and the electric signal is a radiofrequency alternating current and the first and second heating coils are configured to deliver radiofrequency ablative energy to target tissue

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A device for treating varicose veins, comprising:
an elongated catheter comprising:
an elongated shaft defining a longitudinal axis having a proximal end and a distal end, the shaft being sized and configured such that the distal end can be inserted into a target blood vessel;
a heating element disposed near the distal end of the elongated shaft, the heating element comprising a coil member comprising a plurality of first windings about the shaft in a first direction, wherein a plurality of openings in the plurality of first windings are defined along the length of the heating element; and
a plurality of pressure sensors longitudinally spaced from one another along the shaft, wherein each of the pressure sensors is located on the shaft within a respective one of the openings in the plurality of first windings, and wherein adjacent pressure sensors are circumferentially offset from one another, each pressure sensor being configured to generate an output signal indicative of pressure applied thereto by surface of target blood vessel.

2. The device of claim 1, wherein the coil member further comprises a plurality of second windings about the shaft in a second direction different than the first direction, wherein at least some of the plurality of second windings cross over the plurality of first windings at locations spaced along a length of the heating element, and wherein at least some of the openings are defined between the plurality of first windings and the plurality of second windings.

3. The device of claim 2, wherein the plurality of pressure sensors include N pressure sensors; wherein two adjacent pressure sensors of the plurality of pressure sensors are circumferentially offset by an offset degree related to N from one another.

4. The device of claim 1, wherein the plurality of pressure sensors include a first pressure sensor pair and a second sensor pair, wherein the first sensor pair includes a first pressure sensor and a second pressure sensor adjacent to the first pressure sensor, wherein the second pressure sensor is circumferentially offset by a first offset angle from the first pressure sensor, wherein the second sensor pair includes a third pressure sensor and a fourth pressure sensor adjacent to the third pressure sensor, wherein the fourth pressure sensor is circumferentially offset by a second offset angle from the third pressure sensor, wherein the second offset angle is equal to the first offset angle.

5. The device of claim 1, wherein the first and second plurality of windings are arranged to define a plurality of coil segments; wherein adjacent coil segments are longitudinally spaced from one another, defining one or more segment gaps between each adjacent coil segments along a length of the shaft; wherein the device further comprising a temperature sensor; wherein a temperature sensor is disposed within a segment gap of the one or more segments gaps; wherein at least one pressure sensor of the plurality of pressure sensors is disposed in an opening within a coil segment.

6. The device of claim 1, wherein the plurality of pressure sensors include six pressure sensors.

7. The device of claim 6, wherein two adjacent pressure sensors of the plurality of pressure sensors are circumferentially offset by 60 degrees from one another.

8. The device of claim 6, wherein two adjacent pressure sensors of the plurality of pressure sensors are circumferentially offset by 120 degrees from one another.

9. The device of claim 8, wherein the plurality of pressure sensors include at least one selected from a group consisting of a piezoelectric pressure sensor, a capacitive pressure sensor, an inductive pressure sensor, a strain gauge pressure sensor, and a potentiometric pressure sensor.

10. The device of claim 8, wherein the heating element is controlled to deliver ablative energy when an output signal indicative of pressure generated by one pressure sensor of the plurality of pressure sensors is greater than a predetermined threshold.

11. A device for treating varicose veins, comprising:
an energy generator configured to generate an electric signal;
a controller operatively connected to the energy generator to control the generation of the electric signal; and
an elongated catheter connected to the energy generator comprising:
an elongated shaft defining a longitudinal axis having a proximal end and a distal end, the shaft being sized and configured such that the distal end can be inserted into a target blood vessel;
a heating element disposed near the distal end of the elongated shaft, the heating element comprising:
a first coil member comprising a first plurality of windings about the shaft, wherein one or more first openings in the first plurality of windings are defined along a length of the first coil member; and
a second coil member comprising a second plurality of windings about the shaft, wherein one or more second openings in the second plurality of windings are defined along the length of the second coil member; and
a plurality of pressure sensors longitudinally spaced from one another along the shaft, wherein each pressure sensor of the plurality of pressure sensors is located on the shaft within a respective opening of the first openings in the first plurality of windings or the second openings in the second plurality of windings, and wherein at least two adjacent pressure sensors are circumferentially offset from one another, each pressure sensor being configured to generate an output signal indicative of pressure applied thereto by a surface of the target blood vessel;
wherein the first and second coil members are each operatively connected to the energy generator and configured to generate thermal energy when the electric signal generated by the energy generator is delivered thereto.

12. The device of claim 11, wherein the heating element further comprises a third coil member comprising a third plurality of windings about the shaft, wherein one or more third openings in the third plurality of windings are defined along a length of the third coil member; wherein one or more of the plurality of pressure sensors are located on the shaft within one or more of the third openings.

13. The device of claim 11, wherein the controller is configured to adjust the current generated by the energy generator based on the output signal indicative of pressure applied thereto generated by each pressure sensor of the plurality of pressure sensors.

14. The device of claim 13, wherein the controller is configured to control current generated by the energy generator to be selectively delivered to one or both of the first and second coil members.

15. The device of claim 14, further comprising a temperature sensor disposed on the shaft within an opening of the first openings or the second openings, wherein the temperature sensor is longitudinally spaced from one of the plurality of pressures sensors along the shaft.

16. A catheter for use in treating varicose veins, the catheter comprising:
an elongated shaft having a proximal end and a distal end, the shaft being sized and configured such that the distal end can be inserted into a target blood vessel;
a coil member disposed at the distal end of the shaft and comprising a plurality of first windings about the shaft in a first direction, wherein a plurality of openings in the plurality of first windings are defined along the length of the heating element; and
a plurality of pressure sensors longitudinally spaced from one another along the distal end of the shaft, wherein adjacent pressure sensors are circumferentially offset from one another, each pressure sensor being configured to generate an output signal indicative of pressure applied thereto by surface of target blood vessel.

17. The device of claim 16, wherein the coil member further comprises a plurality of second windings about the shaft in a second direction different than the first direction, wherein at least some of the plurality of second windings cross over the plurality of first windings at locations spaced along a length of the heating element.

18. The device of claim 17, wherein the plurality of pressure sensors include N pressure sensors; wherein two adjacent pressure sensors of the plurality of pressure sensors are circumferentially offset by an offset degree related to N from one another.

19. The device of claim 17, wherein the first and second plurality of windings are arranged to define a plurality of coil segments; wherein adjacent coil segments are longitudinally spaced from one another, defining one or more segment gaps between each adjacent coil segments along a length of the shaft; wherein the device further comprising a temperature sensor; wherein a temperature sensor is disposed within a segment gap of the one or more segments gaps; wherein at least one pressure sensor of the plurality of pressure sensors is disposed in an opening within a coil segment.

20. The device of claim 16, wherein the plurality of pressure sensors include a first pressure sensor pair and a second sensor pair, wherein the first sensor pair includes a first pressure sensor and a second pressure sensor adjacent to the first pressure sensor, wherein the second pressure sensor is circumferentially offset by a first offset angle from the first pressure sensor, wherein the second sensor pair includes a third pressure sensor and a fourth pressure sensor adjacent to the third pressure sensor, wherein the fourth pressure sensor is circumferentially offset by a second offset angle from the third pressure sensor, wherein the second offset angle is equal to the first offset angle.
